(19) United States
(12) Patent Application Publication
(10) Pub. No.: US 2007/0060622 A1
(43) Pub. Date: Mar. 15, 2007

(54) Compositions and methods using proton pump inhibitors

(75) Inventor: John R. Ieni, Bloomfield, NJ (US)

Correspondence Address: LAHIVE & COCKFIELD, LLP, ONE POST OFFICE SQUARE, BOSTON, MA, 02109-2127, US

(73) Assignee: EISAI CO., LTD., Tokyo (JP)

(21) Appl. No.: 11/492,971

(22) Filed: Jul. 26, 2006

Related U.S. Application Data
(63) Continuation of application No. PCT/US05/02674, filed on Jan. 31, 2005.
(60) Provisional application No. 60/539,981, filed on Jan. 30, 2004.

(51) Int. Cl. A61K 31/4439; A61K 31/7034; A61K 31/7048; A61K 31/496; A61K 31/573; A61K 31/522; A61K 31/407

(52) U.S. Cl. 514/338; 514/171; 514/263.32; 514/37; 514/253.08; 514/290; 514/28; 514/192; 514/210.09

(57) ABSTRACT

The invention provides methods for treating and preventing cystic fibrosis, radiation therapy-induced emesis, chronic ear infections, bruxism, motion sickness, tooth decay due to emesis and other disorders by administering to a patient a therapeutically effective amount of at least one proton pump inhibitor. In other embodiments, the proton pump inhibitor can be administered with one or more cystic fibrosis drugs, motion sickness drugs, antibiotics, NSAIDs, and migraine drugs.

### RELATED APPLICATIONS

This application is a continuation of PCT/US2005/02674 filed Jan. 31, 2005, which claims priority to U.S. application Ser. No. 60/539,981 filed Jan. 30, 2004.

### FIELD OF THE INVENTION

The invention provides safe and effective methods for treating and preventing gastrointestinal disorders by administering at least one proton pump inhibitor. In one embodiment, the proton pump inhibitor is rabeprazole, a pharmaceutically acceptable salt thereof and/or a stereoisomer thereof.

### BACKGROUND OF THE INVENTION

Peptic ulcers are localized erosions of the mucous membrane of the duodenum and/or stomach, which expose the underlying layers of the gut wall to the acid secretions of the stomach and to the proteolytic enzyme pepsin. Peptic ulceration is a common disease of the gastrointestinal tract and it is estimated that about 10 to 20% of the adult male population will experience peptic ulceration at some time in their lives. ACIPHEX® (Eisai, Inc., Teaneck, N.J.), a proton pump inhibitor, is highly successful in treating peptic ulcers. ACIPHEX® is described in U.S. Pat. No. 5,045,552, the disclosure of which is incorporated by reference herein in its entirety. There is a need in the art for new and improved treatments for peptic ulcers and other gastrointestinal disorders. The invention is directed to these, as well as other, important ends.

### SUMMARY OF THE INVENTION

The invention provides methods for treating and preventing gastrointestinal disorders, exercise-induced gastroesophageal reflux disease, cystic fibrosis, radiation therapy-induced emesis, chronic ear infections, bruxism, motion sickness, tooth decay due to emesis, and other disorders by administering to a patient a therapeutically effective amount of at least one proton pump inhibitor.

In one embodiment, the invention provides a method for treating or preventing a gastrointestinal disorder induced or caused by a nonsteroidal anti-inflammatory drug comprising administering to a patient in need thereof a therapeutically effective amount of at least one proton pump inhibitor and at least one nonsteroidal anti-inflammatory drug selected from the group consisting of diclofenac, celecoxib, rofecoyib, and valdecoxib.

In another embodiment, the invention provides a method for treating cystic fibrosis in a patient in need thereof comprising administering a therapeutically effective amount of at least one proton pump inhibitor and at least one cystic fibrosis drug selected from the group consisting of albuterol, theophylline, ipratropium, guaifenesin, dnase, n-acetylcysteine, triamcinolone, flunisolide, fluticasone, beclomethasone, prednisone, methylprednisone, ibuprofen, montelukast, cromolyn, ciprofloxacin, co-trimoxazole, tobramycin, cephalexin, colistin, dicloxacillin, azithromycin, vitamins, pancrelipase, docusate, casanthranol/docusate, omeprazole, ranitidine, loratadine, cetirizine, and fexofenadine.

In another embodiment, the invention provides a method for treating or preventing radiation therapy induced emesis in a patient in need thereof comprising administering a therapeutically effective amount of at least one proton pump inhibitor.

In another embodiment, the invention provides a method for treating or preventing chronic ear infection in a patient in need thereof comprising administering a therapeutically effective amount of at least one proton pump inhibitor and, optionally, at least one antibiotic.

In another embodiment, the invention provides a method for treating or preventing bruxism in a patient in need thereof comprising administering a therapeutically effective amount of at least one proton pump inhibitor.

In another embodiment, the invention provides a method for treating or preventing gastroesophageal reflux in a patient comprising administering a therapeutically effective amount of at least one proton pump inhibitor prior to anesthesia. The proton pump inhibitor is administered before surgery when the patient is anesthetized; during surgery when the patient is anesthetized; or after surgery when the patient is anesthetized.

In another embodiment, the invention provides a method for treating or preventing motion sickness in a patient in need thereof comprising administering a therapeutically effective amount of at least one proton pump inhibitor and, optionally, at least one motion sickness drug.

In another embodiment, the invention provides a method for treating or preventing migraines in a patient in need thereof comprising administering a therapeutically effective amount of at least one proton pump inhibitor and at least one migraine drug selected from the group consisting of diclofenac, celecoxib, rofecoxib, and valdecoxib.

In another embodiment, the invention provides a method for treating or preventing tooth decay caused by emesis in a patient in need thereof comprising administering a therapeutically effective amount of at least one proton pump inhibitor.

In another embodiment, the invention provides a method for treating gastroesophageal reflux disease in an infant in need thereof comprising administering a therapeutically effective amount of at least one proton pump inhibitor to the infant by iontophoresis.

In another embodiment, the invention provides a method for treating or preventing exercise-induced gastroesophageal reflux disease in a patient in need thereof comprising administering a therapeutically effective amount of at least one proton pump inhibitor.

The invention is described in more detail below.

### DETAILED DESCRIPTION OF THE INVENTION

“Patients” includes animals, preferably mammals, more preferably humans. “Patients” include infants, children and adults, and includes males and females.

The invention provides methods for treating and/or preventing gastrointestinal (GI) disorders in a patient in need thereof by administering at least one proton pump inhibitor and one or more nonsteroidal antiinflammatory drugs (NSAIDs).

In one embodiment the invention provides methods for treating GI disorders induced or caused by NSAIDS in a patient in need thereof by administering at least one proton pump inhibitor and one or more nonsteroidal antiinflammatory drugs (NSAIDs).

In other embodiments of each of these methods, the patient can be administered at least two proton pump inhibitors, where the first proton pump inhibitor is rabeprazole, a stereoisomer thereof and/or a pharmaceutically acceptable salt thereof and where the second proton pump inhibitor is omeprazole, lansoprazole, esomeprazole, pantoprazole, leminoprazole, timoprazole, tenatoprazole, disulprazole, RO 18-5362, IY 81149, 3-butyl-4-(2-methylphenylamino)-8-(2-hydroxyethoxy)-quinoline. The gastrointestinal disorders induced or caused by NSAIDs can be any gastrointestinal disorder known in the art, such as those described herein. In one embodiment, the gastrointestinal disorder is a peptic ulcer or gastrointestinal bleeding. The at least one proton pump inhibitor and at least one NSAID can be administered separately or in the form of a composition.

The term “treating” includes eliminating the gastrointestinal disorder or future re-occurrence thereof, or reducing the severity, duration, and/or symptoms of the gastrointestinal disorder (e.g., compared to an untreated gastrointestinal disorder).

The gastrointestinal disorder can be any known in -.he art. Exemplary gastrointestinal disorders include H. pylori infections, ulcers, erosive esophagitis, gastroesophageal reflux disease (GERD), erosive gastroesophageal reflux disease, gastritis, symptomatic GERD, pregnancy-induced GERD, hypersecretory conditions (e.g., Zollinger-Ellison syndrome, idopathic gastric acid hypersecretion), gastrointestinal motility disorders, Barrett's esophagus, dyspepsia, dysphagia, irritable bowel syndrome, inflammatory bowel disease, infectious enteritis, diarrhea, gastroparesis, collagenous colitis, lymphocytic colitis, short bowel syndrome, bleeding associated with short bowel syndrome, gastrointestinal bleeding, hiatal hernia, emesis, abdominal pain, and the like. “Ulcers” include peptic ulcers, bleeding peptic ulcers, stress ulcers, stomal ulcers, refractory ulcers, esophageal ulcers, fungal-induced ulcers, viral-induced ulcers, and the like. “Peptic ulcers” include gastric ulcers and duodenal ulcers. The ulcers can be associated with H. pylori. Inflammatory bowel disease includes Crohn's disease and ulcerative colitis. Infectious enteritis can be caused, for example, by Campylobacter species, Shigella species, Yersinia species (e.g., Yersinia enterocolitica), Cryptosporidium species, Giardia species (e.g., Giardia lamblia), Salmonella species, Pseudomonas species (e.g., Pseudomonas aeruginosa), and the like.

Any NSAID can be used in the compositions and methods of the invention. NSAIDs include COX-1 and/or COX-2 inhibitors. Exemplary COX-2 inhibitors include celecoxib, rofecoxib, valdecoxib, and the like. Exemplary NSAIDs include celecoxib, rofecoxib, valdecoxib, ibuprofen, acetaminophen, aspirin, naproxen, acetaminophen/aspirin/caffeine, ketorolac, ketoprofen, diflunisal, salsalate, salicylate, salicylamide, thiosalicylate, trisalicylate, mesalamine, sulfasalazine, methylsalicylate, phenylbutazone, oxyphenbutazone, antipyrine, aminopyrine, dipyrone, azapropazone, phenacetin, indomethacin, sulindac, mefenamic, meclofenamic, flufenamic, tolfenamic, etofenamic, tolmetin, naproxen, flurbiprofen, fenoprofen, fenbufen, pirprofen, oxaprozin, indoprofen, tiaprofenic acid, piroxicam, ampiroxicam, tenoxicam, tolmetin, meloxicam, tenidap, diclofenac, diclofenaclmisoprostol, sulindac, etodolac, nabumentone, and the like.

In one embodiment, the invention provides methods for treating and preventing gastrointestinal disorders by administering to a patient in need thereof a therapeutically effective amount of at least one proton pump inhibitor and celecoxib. In one embodiment, the proton pump inhibitor is rabeprazole, a pharmaceutically acceptable salt thereof and/or a stereoisomer thereof. In another embodiment, the invention provides methods for treating and preventing gastrointestinal disorders by administering to a patient in need thereof a therapeutically effective amount of at least one proton pump inhibitor and rofecoxib. In one embodiment, the proton pump inhibitor is rabeprazole, a pharmaceutically acceptable salt thereof and/or a stereoisomer thereof. In another embodiment, the invention provides methods for treating and preventing gastrointestinal disorders induced or caused by NSAIDs by administering to a patient in need thereof a therapeutically effective amount of at least one proton pump inhibitor and valdecoxib. In one embodiment, the proton pump inhibitor is rabeprazole, a pharmaceutically acceptable salt thereof and/or a stereoisomer thereof. In another embodiment, the invention provides methods for treating and preventing gastrointestinal disorders induced or caused by NSAIDs by administering to a patient in need thereof a therapeutically effective amount of at least one proton pump inhibitor and diclofenac. In one embodiment, the proton pump inhibitor is rabeprazole, a pharmaceutically acceptable salt thereof and/or a stereoisomer thereof.

The invention provides methods for treating and/or preventing exercise-induced GERD in a patient in need thereof by administering a therapeutically effective amount of at least one proton pump inhibitor. It has been discovered that exercise, such as vigorous or jarring exercises, can cause GERD. In this embodiment of the invention, a patient can be administered at least one proton pump inhibitor before, during and/or after exercise to prevent or treat GERD caused by exercising.

The invention provides methods for treating cystic fibrosis by administering to a patient in need thereof a therapeutically effective amount of at least one proton pump inhibitor and at least one cystic fibrosis drug. Cystic fibrosis is a disorder of the cells that line the lungs, small intestines, sweat glands and pancreas, where mucus contributes to the destruction of lung tissue. Symptoms of cystic fibrosis include excessive appetite, poor weight gain, diarrhea, persistent cough, and other digestive disorders. The proton pump inhibitor is administered to improve the pH of the gastrointestinal environment by reducing the gastric acid output. The patient can be an adult or a child.

Cystic fibrosis drugs include, for example, bronchodilators, mucolytics, anti-inflammatives, antibiotics, vitamins, pancreatic enzymes, stool softeners, GI drugs, antihistamines, and nasal sprays. Exemplary cystic fibrosis drugs include albuterol, theophylline, ipratropium, guaifenesin, dnase, n-acetylcysteine, triamcinolone, flunisolide, fluticasone, beclomethasone, prednisone, methylprednisone, ibuprofen, montelukast, cromolyn, ciprofloxacin, co-trimoxazole, tobramycin, cephalexin, colistin, dicloxacillin, azithromycin, vitamins, pancrelipase, docusate, casanthranol and docusate, omeprazole, ranitidine, loratadine, cetirizine, fexofenadine, and the like.

The invention provides methods for treating and preventing radiation therapy-induced emesis by administering to a patient in need thereof a therapeutically effective amount of at least one proton pump inhibitor. The proton pump inhibitor can be administered before, during and/or after radiation therapy. Radiation therapy-induced emesis is an acute event appearing from 30 minutes to 4 hours following irradiation. Emetogenic risk is high for total body irradiation, upper abdomen and hemibody irradiation, moderate for thorax and pelvic irradiation, and low for radiotherapy to the head and neck, brain, skin and extremities. Radiation therapy-induced emesis results from a complex interaction between various neurotransmitters and receptors in the CNS and gastrointestinal tract.

The invention provides methods for treating and/or preventing chronic ear infections by administering to a patient in need thereof a therapeutically effective amount of at least one proton pump inhibitor and, optionally, at least one antibiotic. A chronic ear infection may be the result of an acute ear infection that does not clear completely, or the result of recurrent ear infections. Acute otitis media (acute middle ear infection), most common in children, occurs when there is bacterial or viral infection of the fluid of the middle ear, causing the production of pus or excess fluid. This may be accompanied by bleeding in the middle ear. Inflammation of the ear (sterile or serous otitis) may occur when there is a collection of fluid in the ear that is not infected. This may be caused by overproduction of fluid by the structures in the middle ear or by blockage of the drainage system (the eustachian tube). Pressure from fluids associated with ear infection may cause the eardrum to rupture. A ruptured eardrum can also result in ear infection by allowing bacteria or viruses direct entry to the middle ear. Ear infections are often associated with respiratory infections or with blocked sinuses or eustachian tubes caused by allergies or enlarged adenoids. A chronic ear infection may spread into the mastoid bone (mastoiditis), or pressure from fluid build-up may rupture the eardrum or damage the bones of the middle ear. A chronic ear infection may be more destructive than an acute ear infection because its effects are prolonged or repeated, and it may cause permanent damage to the ear. Ear infections are more common in children because their eustachian tubes are shorter, narrower, and more horizontal than in adults. In one embodiment, the patient is an adult. In another embodiment, the patient is a child.

Any antibiotic can be used in the compositions and methods of the invention. Exemplary antibiotics include amoxicillin, amoxicillin and clavulanate potassium, cefpodoxime proxetil, ceftriaxone, cefuroxime, trimethoprim/sulfamethoxazole, and the like.

In other embodiments, the invention provides methods for preventing gastroesophageal reflux during and/or after surgery by administering to a patient in need thereof a therapeutically effective amount of at least one proton pump inhibitor prior to the administration of anesthesia.

In other embodiments, the invention provides methods for treating and preventing bruxism by administering to a patient in need thereof a therapeutically effective amount of at least one proton pump inhibitor. Bruxism, commonly known as tooth grinding, is the clenching together of the bottom and upper jaw accompanied by the grinding of the lower set of teeth with the upper set.

In other embodiment, the invention provides methods for treating and/or preventing motion sickness by administering to a patient in need thereof a therapeutically effective amount of at least one proton pump inhibitor. In another embodiment, the invention provides methods for preventing and treating motion sickness by administering a therapeutically effective amount of at least one proton pump inhibitor and at least one motion sickness drug. The proton pump inhibitor and motion sickness drug can be administered separately or in the form of a composition. Motion sickness applies to seasickness, carsickness, airsickness or sickness caused by anything else such as a swing or fairground amusements. Exemplary motion sickness drugs include scopolamine, promethazine, dimenhydrinate, diphenhydramine, cyclizine, buclizine, and meclizine, and the like.

In another embodiment, the invention provides methods for preventing and treating migraines by administering a therapeutically effective amount of at least one proton pump inhibitor and at least one non-steroidal antiinflammatory drugs (NSAIDs).

The migraines can be classic migraines, common migraines, complicated migraines, and/or cluster headaches. In other embodiments, the migraines can be menstrual migraines, premenstrual migraines, ophthalmic migraines, and/or ophthalmoplegic migraines. In other embodiments, the migraines can be fulgurating migraines, Harris' migraines, and/or hemiplegic migraines. In still other embodiments, the migraines can be abdominal migraines.

In another embodiment, the invention provides methods for preventing and treating tooth decay caused due to emesis by administering a therapeutically effective amount of at least one proton pump inhibitor. The excessive reflux experienced by patients with GERD overwhelms their intrinsic mucosal defense mechanisms, resulting in many symptoms. The most common symptom of GERD is heartburn. Besides the discomfort of heartburn, reflux results in symptoms of esophageal inflammation, such as odynophagia (pain on swallowing) and dysphagia (difficult swallowing). The acid reflux may also cause oral symptoms such as tooth enamel decay, gingivitis, halitosis, and waterbrash and throat symptoms such as soreness, laryngitis, hoarseness, and a globus sensation; and earache. The effects of GERD on the mouth and salivary glands result in symptoms of waterbrush, gingivitis, and tooth decay. Waterbrash is the spontaneous appearance of high volumes of saliva in the mouth, and is caused by a vagally mediated reflex initiated by acid in the esophagus. Gingivitis and tooth decay are caused by contact with acidic refluxate.

Any proton pump inhibitor can be used in the compositions and methods described herein. Exemplary proton pump inhibitors include rabeprazole, omeprazole, lansoprazole, esomeprazole, pantoprazole, leminoprazole, timoprazole, tenatoprazole, disulprazole, RO 18-5362, IY 81149, 3-butyl-4-(2-methylphenylamino)-8-(2-hydroxyethoxy)-quinoline, and the like.

In one embodiment, the proton pump inhibitors are compounds of formula (I), pharmaceutically acceptable salts thereof, and/or stereoisomers thereof:

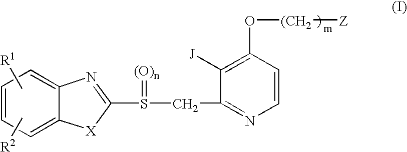

wherein R¹ and R² are each independently a hydrogen atom, a halogen atom, a lower alkyl, lower alkoxy, halogenated lower alkyl, lower alkoxycarbonyl or carboxyl group; X is —O—, —S— or ═N—R³, wherein R³ is a hydrogen atom or a lower alkyl, phenyl, benzyl or lower alkoxycarbonyl group; and Z is: (1) —O(CH₂)ₚ—O—R⁴, wherein p is an integer of 1 to 3 and R⁴ is hydrogen atom or a lower alkyl, aryl or aralkyl group; (2) —O—(CH₂)_{q}—R⁵, wherein q is an integer of 1 to 3 and R⁵ is a halogen atom or an alkoxycarbonyl, aryl or heteroaryl group; (3) —O—(CH₂)ᵣ—O—(CH₂)ₛ—O—R⁶, wherein r and s are each independently an integer of 1 to 5 and R⁶ is a hydrogen atom or a lower alkyl group; (4) 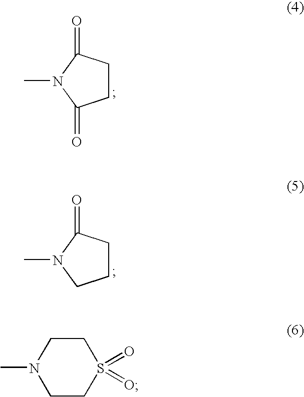
(7) —S(O)ₜ-A, wherein t is an integer of 0 to 2, and A is a lower alkyl, alkoxycarbonylmethyl, pyridyl, furyl, 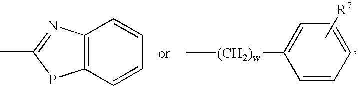
wherein B is —NH—, —O— or —S—, and w is an integer of 0 or 1; (8) —N(R⁸)—CH₂—C₆H₅, wherein R⁸ is an acetoxy or lower alkyl group; (9) —OR⁹, wherein R⁹ is a hydrogen atom, a lower alkyl or aryl group; n is an integer of 0 to 2; m is an integer of 2 to 10, and J and K are each independently a hydrogen atom or a lower alkyl group, with the proviso that when Z is a group falling under the above category (9), then R⁹ is a lower alkyl group and m stands for an integer of 3 to 10, and pharmaceutically acceptable salts thereof.

The same definitions for R¹, R², X, n, J, K, Z and m are used throughout the specification that follows and in the appended claims.

Also disclosed are pharmaceutical compositions containing one or more of these compounds as the active ingredient(s) in a pharmaceutically acceptable carrier, adjuvant or vehicle.

In the definition of the compounds of formula (I), the lower alkyl group defined with respect to R¹, R², R³, R⁴, R⁶, R⁷, R⁸, A, J and K can be a straight-chain or branched alkyl group having 1 to 6 carbon atoms. Examples include methyl, ethyl, n-propyl, n-butyl, isopropyl, isobutyl, 1-methylpropyl, tert-butyl, n-pentyl, 1-ethylpropyl, isoamyl and n-hexyl groups, among which methyl and ethyl groups are most preferred.

The lower alkoxy group and the lower alkoxy moiety of the lower alkoxycarbonyl group defined above with respect to R¹ and R² can be an alkoxy group derived form the above lower alkyl group. Methoxy and ethoxy groups are most preferred.

The halogen atom defined above includes chlorine, bromine, iodine or fluorine. The aryl group defined above with respect to R⁴ and R⁵ can be phenyl, tolyl, xylyl, napthyl or the like, which can be substituted with a lower alkoxy or hydroxyl group, a halogen atom or the like.

Examples of the arylalkyl defined above with respect to R⁴ include benzyl and phenethyl groups.

Examples of the heteroaryl group defined above with respect to R⁵ include pyridyl and furyl groups.

In the definition of Z in formula (I), groups 1, 2, 3, 4, 5 and 9 are preferred; and group 9 is the most preferred. As for R¹ and R², hydrogens for both and then a combination of a lower alkyl (e.g., methyl) for R¹ and hydrogen for R² are preferred. X is preferably ═NR³, where R³ is hydrogen. A preferred value for n is 1. The preferred substituents for J and K are both hydrogen or where J is lower alkyl (e.g., methyl), and K is hydrogen, or when J is hydrogen and K is lower alkyl (e.g., methyl). Thus, J or K are independently preferably hydrogen or methyl, most preferably J is methyl and K is hydrogen.

In another embodiment, the compounds of formula (I) are compounds of formula (A), pharmaceutically acceptable salts thereof, and/or stereoisomers thereof: 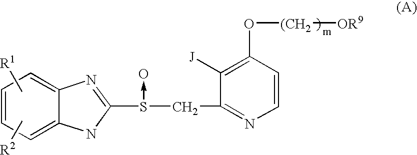
wherein R¹, R², J, m and R⁹ have the same meanings as defined above.

In formula (A), the preferred R¹ and R² substituents are both hydrogen, or R¹ is 5-lower alkoxy, 5-lower alkyl or 5-halogenated lower alkyl and R² is hydrogen. The preferred substituent for J is hydrogen or methyl; the preferred value for m is in the range of 3 to 10, the most preferred being 3; and the preferred R⁹ substituent is lower alkyl (e.g., methyl), or aryl. Among these possibilities for the compounds of formula (A), the preferred combination is when R¹ and R² are both hydrogen, J is methyl, m is 3 and R⁹ is methyl.

Another group of preferred compounds in formula (A) are combinations of the above substituents where both R¹ and R² are hydrogen, J is hydrogen, m is 3 and R⁹ is methyl.

Another group of preferred compounds falling within formula (A) is when both R¹ and R² are hydrogen, J is methyl, m is 2 and R⁹ is benzyl.

In another embodiment, the compounds of formula (I) are compounds of formula (B), pharmaceutically acceptable salts thereof, and/or stereoisomers thereof:

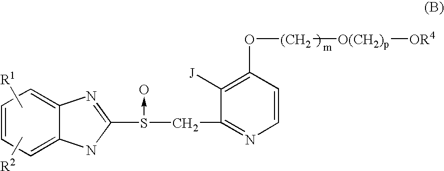

wherein R¹, R², J, p, m and R⁴ have the same meanings as given above.

In formula (B), the preferred substituents for R¹ and R² are both hydrogen; or when R¹ is 5-lower alkoxy, 5-lower alkyl or 5-halogenated lower alkyl, R² is hydrogen. The preferred value of m is 2 or 3; the preferred value for p is 2 or 3; and the preferred substituent for R⁴ is methyl or benzyl. Of the above possibilities for formula (B), the most preferred combination is where R¹ is 5-methyl, R² is hydrogen, J is methyl, m is 2, p is 2 and R⁴ is methyl.

In another embodiment, the compound of formula I is a compound of formula (C), a pharmaceutically acceptable salt thereof, and/or a stereoisomer thereof: 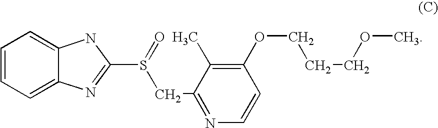
Preferably, the compound of formula (C) is a sodium salt, which is known as rabeprazole sodium or ACIPHEX® (Eisai Inc., Teaneck, N.J.).

Although the compounds of the invention can be present as a hydrate or as a stereoisomer, the hydrates and stereoisomers are included within the scope of the invention. For example, the compound of formula (C) can be a compound of formula (D) or a pharmaceutically acceptable salt thereof (e.g., a sodium salt): 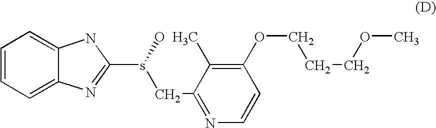
The compound of formula (D) is R (+) rabeprazole.

Alternatively, the compound of formula (C) can be a compound of formula (E) or a pharmaceutically acceptable salt thereof (e.g., a sodium salt): 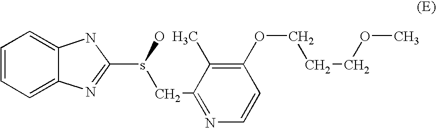
The compound of formula (E) is S (−) rabeprazole.

The compounds of the invention can be administered as any pharmaceutically acceptable salt known in the art. Pharmaceutically acceptable salts are known in the art and include those of inorganic acids, such as hydrochloride, sulfate, hydrobromide, sulfate, and phosphate; those of organic acids, such as formate, acetate, maleate, tartrate, trifluoroacetate, methanesulfonate, benzenesulfonate and toluenesulfonate, and those of amino acids such as arginine, aspartic acid and glutamic acid. When certain substituents are selected, the compounds of the invention can form, for example, alkali metal salts, such as sodium or potassium salts; alkaline earth metal salts, such as calcium or magnesium salts; organic amine salts, such as a salt with trimethylamine, triethylamine, pyridine, picoline, dicyclohexylamine or N,N′-dibenzylethylenediamine. One skilled in the art will recognize that the compounds of the invention can be made in the form of any of these or of any other pharmaceutically acceptable salt. For example, compounds represented by formula (I), wherein X is ═N—R³ and R³ is a hydrogen atom, or compounds represented by formula (I), wherein Z is a group falling under the category 7 and B is a group of —NH—, can be present as a metal salt, such as sodium, potassium, magnesium or calcium.

The proton pump inhibitors are commercially available or can be prepared by processes known in the art. Rabeprazole sodium is commercially available as ACIPHEX® from Eisai Inc., Teaneck, N.J., and is described, for example, in U.S. Pat. No. 5,045,552, the disclosure of which is incorporated by reference herein in its entirety. Methods for preparing R (+) rabeprazole are described in WO 99/55157, the disclosure of which is incorporated by reference herein in its entirety. Methods for preparing S (−) rabeprazole are described in WO 99/55158, the disclosure of which is incorporated by reference herein in its entirety.

A therapeutically effective dosage regimen for treating the diseases described herein with the proton pump inhibitors and/or antibiotics is selected in accordance with a variety of factors, including the age, weight, sex, and medical condition of the patient, the severity of the disease, the route of administration, pharmacological considerations such as the activity, efficacy, pharmacokinetic and toxicology profiles of the particular proton pump inhibitor and/or antibiotics, whether a drug delivery system is used and whether the proton pump inhibitor and/or antibiotics is administered as part of a drug combination.

When administered separately, the proton pump inhibitors and/or other drugs described herein (e.g., NSAIDs, antibiotics, motion sickness drugs, cystic fibrosis drugs) can be administered about the same time as part of an overall treatment regimen, i.e., as a combination therapy or drug cocktail. “About the same time” includes administering the proton pump inhibitor and/or other drugs described herein (e.g., NSAIDs, antibiotics, motion sickness drugs, cystic fibrosis drugs) at the same time, at different times on the same day, or on different days, as long as they are administered as part of an overall treatment regimen.

The proton pump inhibitors can be administered in amounts of about 0.01 to about 200 mg per day, preferably about 0.05 to about 50 mg per day, more preferably about 0.1 to about 40 mg per day, still more preferably about 10 to about 30 mg per day, still more preferably about 10 to about 20 mg per day. The compounds and/or compositions can be administered once a day or in divided doses, for example from 2 to 4 times a day, preferably once per day. One skilled in the art will recognize that when the compounds and/or compositions of the invention are administered to infants or children, the dose can be smaller than the dose administered to adults, and that the dose can be dependent upon the size and weight of the patient.

In preferred embodiments of the methods described herein, rabeprazole sodium, which is commercially available as ACIPHEX® (Eisai Inc., Teaneck, N.J.), is administered as a tablet containing 10 or 20 milligrams rabeprazole sodium. The tablets can be administered one to about four times a day. In preferred embodiments, one 20 milligram ACIPHEX® tablet is administered once a day for the methods described herein. One skilled in the art will appreciate that when rabeprazole sodium is administered to infants or children, the dose can be smaller than the dose that is administered to adults.

The proton pump inhibitors and/or antibiotics can be administered orally, topically, parenterally, by inhalation (nasal, oral, aural), or rectally in dosage unit formulations containing conventional nontoxic pharmaceutically acceptable carriers, adjuvants, and vehicles as desired. The term parenteral as used herein includes subcutaneous, intravenous, intramuscular, intrathecal, intrasternal injection, or infusion techniques. Preferably, the proton pump inhibitors are orally administered as tablets.

Injectable preparations, for example, sterile injectable aqueous or oleaginous suspensions can be formulated according to the known art using suitable dispersing or wetting agents, suspending agents (e.g., methylcellulose, Polysorbate 80, hydroxyethylcellulose, acacia, powdered tragacanth, sodium carboxymethylcellulose, polyoxytehylene sorbitan monolaurate and the like), pH modifiers, buffers, solubilizing agents (e.g., polyoxyethylene hydrogenated castor oil, Polysorbate 80, nicotinamide, polyoxyethylene sorbitan monolaurate, Macrogol, an ethyl ester of castor oil fatty acid, and the like), preservatives and/or stabilizers. The sterile injectable preparation can also be a sterile injectable solution or suspension in a nontoxic parenterally acceptable diluent or solvent, for example, as a solution in 1,3-butanediol. Among the acceptable vehicles and solvents that can be used are water, Ringer's solution, and isotonic sodium chloride solution. In addition, sterile, fixed oils are conventionally used as a solvent or suspending medium. For this purpose any bland fixed oil can be used including synthetic mono- or diglycerides, in addition, fatty acids such as oleic acid find use in the preparation of injectables. The preparations can be lyophilized by methods known in the art.

Solid dosage forms for oral administration can include capsules, tablets, sublingual tablets, powders, granules, gels, effervescent tablets, effervescent wafers, effervescent capsules, and effervescent powders; most preferably tablets. The solid dosage form can be a solid microencapsulated dosage, such as a microencapsulated powder, microencapsulated granules or a microencapsulated gel. A solid dosage form for oral administration can be prepared by mixing an active principle with filler and, if necessary, binder, disintegrating agent, lubricant, coloring agent, corrigent or the like and converting the obtained mixture into a tablet, coated tablet, granule, powder or capsule. Examples of the filler include lactose, corn starch, sucrose, glucose, sorbitol, crystalline cellulose and silicon dioxide, while those of the binder include polyvinyl alcohol, polyvinyl ether, ethylcellulose, methylcellulose, acacia, tragacanth, gelatin, shellac, hydroxypropylcellulose, hydroxypropylstarch and polyvinylpyrrolidone. Examples of the disintegrating agent include starch, agar, gelatin powder, crystalline cellulose, calcium carbonate, sodium hydrogencarbonate, calcium citrate, dextrin and pectin, while those of the lubricant include magnesium stearate, talc, polyethylene glycol, silica and hardened vegetable oils. The coloring agent can be any one which is permitted to be added to drugs. Examples of the corrigent include cacao powder, mentha herb, aromatic powder, mentha oil, borneol and powdered cinnamon bark. The tablets and granules can be, if necessary, coated with sugar, gelatin or the like. Preferably, the tablets have an enteric coating.

The proton pump inhibitor can be formulated or admixed with, for example, an acid (e.g., citric acid) and a bicarbonate (e.g., sodium bicarbonate) to form an effervescent tablet, capsule, wafer or powder. After addition of the effervescent tablet, capsule, wafer or powder to a liquid (e.g., water, juice) a bicarbonate solution of the proton pump inhibitor is formed.

In other embodiments, the solid dosage form can be packaged as granules or a powder in a pharmaceutically acceptable carrier, where the granules or powder are removed from the packaging and sprinkled on food or mixed with a liquid, such as water or juice. In this embodiment, the active compound can be mixed with flavoring or sweetening agents. The packaging material can be plastic, polyester films, nylon films, polyolefin films, shrink packing films, coated paper, or any material that prevents water or moisture from reaching the granules and/or powder.

Liquid dosage forms for oral administration can include pharmaceutically acceptable emulsions, solutions, suspensions, and syrups containing inert diluents commonly used in the art, such as water. The liquid dosage form can be a microencapsulated liquid, including microencapsulated emulsions, microencapsulated solutions, microencapsulated suspensions and microencapsulated syrups. Such compositions can also comprise adjuvants, such as wetting agents, emulsifying and suspending agents, and sweetening, flavoring, and perfuming agents.

In another embodiment, the invention provides compositions comprising at least one proton pump inhibitor and at least one cyclodextrin or a cyclodextrin derivative. The compositions can be in the form of a sachet, granules, micro-pellets, or beads. Cyclodextrin derivatives are described, for example, in U.S. Pat. No. 3,459,731, EP-A-149,197, EP-A-197,571, U.S. Pat. No. 4,535,152 or WO 90/12035. Cyclodextrin derivatives include alpha-cyclodextrins, beta-cyclodextrins, and gamma-cyclodextrins. The cyclodextrins can be ethers and/or mixed ethers thereof wherein one or more of the hydroxy groups of the anhydroglucose units of the cyclodextrin are substituted with C₁₋₆ alkyl (e.g., methyl, ethyl or isopropyl); hydroxy C₁₋₆ alkyl (e.g., hydroxyethyl, hydroxypropyl or hydroxybutyl); carboxy C₁₋₆ alkyl (e.g., carboxymethyl or carboxyethyl); C₁₋₆ alkyl-carbonyl (e.g., acetyl); C₁₋₆ alkyloxycarbonyl C₁₋₆ alkyl or carboxy C₁₋₆ alkyl-oxy C₁₋₆ alkyl (e.g., carboxymethoxypropyl or carboxyethoxypropyl); C₁₋₆ alkylcarbonyloxy C₁₋₆ alkyl (e.g., 2-acetyloxypropyl). In one embodiment, complexants and/or solubilizers for the proton pump inhibitors are beta-cyclodextrin; 2,6-dimethyl-beta-cyclodextrin, 2-hydroxyethyl-beta-cyclodextrin, 2-hydroxyethyl-gamma-cyclodextrin, 2-hydroxypropyl-gamma-cyclodextrin and (2-carboxy-methoxy)propyl-beta-cyclodextrin. and in particular 2-hydroxypropyl-beta-cyclodextrin. In another embodiment, the cyclodextrin is beta-cyclodextrin.

For administration by aural, oral or nasal inhalation, the compounds and compositions can be delivered from an insufflator, a nebulizer or a pressured pack or other convenient mode of delivering an aerosol spray. Pressurized packs can include a suitable propellant. Alternatively, for administration by aural, oral or nasal inhalation, the compounds and compositions can be administered in the form of a dry powder composition or in the form of a liquid spray.

Suppositories for rectal administration can be prepared by mixing one or more compounds or compositions with suitable nonirritating excipients, such as cocoa butter and/or polyethylene glycols, that are solid at room temperature and that melt at body temperature. Alternatively, enemas can be used to for rectal administration of the active compounds.

For topical administration to the epidermis, the proton pump inhibitors can be formulated as ointments, creams or lotions, or as the active ingredient of a transdermal patch. The compounds and compositions can also be administered via iontophoresis or osmotic pump. Ointments, creams and lotions can be formulated with an aqueous or oily base with the addition of suitable thickening and/or gelling agents. Alternatively, ointments, creams and lotions can be formulated with an aqueous or oily base and can also contain one or more emulsifying agents, stabilizing agents, dispersing agents, suspending agents, thickening agents, and/or coloring agents. As creams or lotions, the proton pump inhibitors can be mixed to form a smooth, homogeneous cream or lotion with, for example, one or more of a preservative (e.g., benzyl alcohol 1% or 2% (wt/wt)), emulsifying wax, glycerin, isopropyl palmitate, lactic acid, purified water, sorbitol solution. Such topically administrable compositions can contain polyethylene glycol 400. To form ointments, the proton pump inhibitors can be mixed with one or more of a preservative (e.g., benzyl alcohol 2% (wt/wt)), petrolatum, emulsifying wax, and Tenox (II) (e.g., butylated hydroxyanisole, propyl gallate, citric acid, propylene glycol). Woven pads or rolls of bandaging material, e.g., gauze, can be impregnated with the transdermally administrable compositions for topical application.

The proton pump inhibitors can also be topically applied using a transdermal system, such as one of an acrylic-based polymer adhesive with a resinous crosslinking agent impregnated with the proton pump inhibitors and laminated to an impermeable backing. For example, the proton pump inhibitors can be administered in the form of a transdermal patch, such as a sustained-release transdermal patch. Transdermal patches can include any conventional form such as, for example, an adhesive matrix, a polymeric matrix, a reservoir patch, a matrix- or monolithic-type laminated structure, and are generally comprised of one or more backing layers, adhesives, penetration enhancers, and/or rate-controlling membranes. Transdermal patches generally have a release liner, which is removed to expose the adhesive/active ingredient(s) prior to application. Transdermal patches are described in, for example, U.S. Pat. Nos. 5,262,165, 5,948,433, 6,010,715 and 6,071,531, the disclosures of which are incorporated by reference herein in their entirety.

The proton pump inhibitors can also be topically applied using a transdermal system, such as inotophoresis system. lontophoresis system is a transdermal delivery system in which a substance bearing a charge (e.g., a proton pump inhibitor) is propelled through the skin by a low electrical current. This method can be used to drive a drug across the skin barrier, as is done with pilocarpine to stimulate sweating in the sweat chloride test for cystic fibrosis. Iontophoresis is an effective and painless method of delivering medication to a localized tissue area, in an infant, by applying electrical current to a solution of the medication. The application of a positive current will drive positively charged drug molecules away from the electrode and into the tissues. In one embodiment, iontophoresis is used to deliver proton pump inhibitors to infants for treating and/or preventing GI disorders. In one embodiment, the gastrointestinal disorder is GERD.

The invention provides for the proton pump inhibitors and, optionally, other active ingredients, to be administered nasally to a patient to treat the diseases and disorders described herein and those described, for example, in PCT Application No. PCT/US02/36857, the disclosure of which is incorporated by reference herein in its entirety. “Administered nasally” or “nasal administration” is intended to mean that at least one proton pump inhibitor is combined with a suitable delivery system for absorption across the nasal mucosa of a patient, preferably a human.

The proton pump inhibitors of the invention can be administered, for example, as nasal sprays, nasal drops, nasal suspensions, nasal gels, nasal ointments, nasal creams or nasal powders. The proton pump inhibitors can also be administered using nasal tampons or nasal sponges. The proton pump inhibitors of the invention can be brought into a viscous basis via systems conventionally used, for example, natural gums, methylcellulose and derivatives, acrylic polymers (carbopol) and vinyl polymers (polyvinylpyrrolidone). In the compositions, many other excipients known in the art can be added such as water, preservatives, surfactants, solvents, adhesives, antioxidants, buffers, bio-adhesives, viscosity enhancing agents and agents to adjust the pH and the osmolarity.

The nasal delivery systems can take various forms including aqueous solutions, non-aqueous solutions and combinations thereof. Aqueous solutions include, for example, aqueous gels, aqueous suspensions, aqueous liposomal dispersions, aqueous emulsions, aqueous microemulsions and combinations thereof. Non-aqueous solutions include, for example, non-aqueous gels, non-aqueous suspensions, non-aqueous liposomal dispersions, non-aqueous emulsions, non-aqueous microemulsions and combinations thereof.

In other embodiments, the nasal delivery system can be a powder formulation. Powder formulations include, for example, powder mixtures, powder microspheres, coated powder microspheres, liposomal dispersions and combinations thereof. Preferably, the powder formulation is powder microspheres. The powder microspheres are preferably formed from various polysaccharides and celluloses selected from starch, methylcellulose, xanthan gum, carboxymethylcellulose, hydroxypropyl cellulose, carbomer, alginate polyvinyl alcohol, acacia, chitosans, and mixtures of two or more thereof.

In certain embodiments, the particle size of the droplets of the aqueous and/or non-aqueous solution or of the powders delivered to the nasal mucosa can be, for example, about 0.1 micron to about 100 microns; from about 1 micron to about 70 microns; from about 5 microns to about 50 microns; or from about 10 microns to about 20 microns. The particle sizes can be obtained using suitable containers or metering devices known in the art. Exemplary devices include mechanical pumps in which delivery is made by movement of a piston; compressed air mechanisms in which delivery is made by hand pumping air into the container; compressed gas (e.g., nitrogen) techniques in which delivery is made by the controlled release of a compressed gas in the sealed container; liquefied propellant techniques in which a low boiling liquid hydrocarbon (e.g., butane) is vaporized to exert a pressure and force the composition through the metered valve; and the like. Powders may be administered, for example, in such a manner that they are placed in a capsule that is then set in an inhalation or insufflation device. A needle is penetrated through the capsule to make pores at the top and the bottom of the capsule and air is sent to blow out the powder particles. Powder formulation can also be administered in a jet-spray of an inert gas or suspended in liquid organic fluids.

In one embodiment, the invention provides a nasally administrable pharmaceutical composition comprising at least one proton pump inhibitor dispersed in a nasal delivery system that improves the solubility of the proton pump inhibitor. The nasal delivery system that improves solubility can include one of the following or combinations thereof: (i) a glycol derivative (e.g., propylene glycol, polyethylene glycol, mixtures thereof); (ii) a sugar alcohol (e.g., mannitol. xylitol, mixtures thereof); (iii) glycerin; (iv) a glycol derivative (e.g., propylene glycol, polyethylene glycol or mixtures thereof) and glycerin; (v) ascorbic acid and water; (vi) sodium ascorbate and water; or (vii) sodium metabisulfite and water.

In another embodiment, the invention provides a nasally administrable pharmaceutical composition comprising at least one proton pump inhibitor and a nasal delivery system, where the nasal delivery system comprises at least one buffer to maintain the pH of the proton pump inhibitor, at least one pharmaceutically acceptable thickening agent and at least one humectant. The nasal delivery system can optionally further comprise surfactants, preservatives, antioxidants, bio-adhesives, pH adjusting agents, isotonicity agents, solubilizing agents, and/or other pharmaceutically acceptable excipients. The proton pump inhibitor can optionally be dispersed in a nasal delivery system that improves its solubility.

In another embodiment, the invention provides a nasally administrable pharmaceutical composition comprising at least one proton pump inhibitor and a nasal delivery system, where the nasal delivery system comprises at least one solubilizing agent, at least one pharmaceutically acceptable thickening agent and at least one humectant. The nasal delivery system can optionally further comprise buffers, pH adjusting agents, isotonicity agents, surfactants, preservatives, antioxidants, bio-adhesives, and/or other pharmaceutically acceptable excipients. The proton pump inhibitor can optionally be dispersed in a nasal delivery system that improves its solubility.

In another embodiment, the invention provides a nasally administrable pharmaceutical composition comprising at least one proton pump inhibitor and a nasal delivery system, where the nasal delivery system comprises at least one buffer to maintain the pH of the proton pump inhibitor, at least one pharmaceutically acceptable thickening agent, at least one humectant, and at least one surfactant. The nasal delivery system can optionally further comprise pH adjusting agents, isotonicity agents, solubilizing agents, preservatives, antioxidants, bio-adhesives, and/or other pharmaceutically acceptable excipients. The proton pump inhibitor can optionally be dispersed in a nasal delivery system that improves its solubility.

In yet another embodiment, the invention provides a nasally administrable pharmaceutical composition comprising at least one proton pump inhibitor and a nasal delivery system, where the nasal delivery system comprises at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The proton pump inhibitor can optionally be dispersed in a nasal delivery system that improves its solubility.

In yet another embodiment, the invention provides a nasally administrable pharmaceutical composition comprising at least one proton pump inhibitor and a nasal delivery system, where the nasal delivery system comprises at least one buffer to maintain the pH of the proton pump inhibitor, at least one pharmaceutically acceptable thickening agent, at least one humectant, at least one surfactant, and at least one solubilizing agent. The nasal delivery system can optionally further comprise buffers, pH adjusting agents, isotonicity agents, preservatives, antioxidants, bio-adhesives, and/or other pharmaceutically acceptable excipients. The proton pump inhibitor can optionally be dispersed in a nasal delivery system that improves its solubility.

The nasally administrable pharmaceutical compositions of the invention preferably provide a peak plasma concentration of the proton pump inhibitor in less than one hour, preferably within about 5 minutes to about 30 minutes, more preferably within about 5 minutes to about 20 minutes, after administration to the patient.

The buffer has a pH that is selected to optimize the absorption of the proton pump inhibitor across the nasal mucosa. The particular pH of the buffer can vary depending upon the particular nasal delivery formulation as well as the specific proton pump inhibitor selected. Buffers that are suitable for use in the invention include acetate (e.g., sodium acetate), citrate (e.g., sodium citrate dihydrate), phthalate, borate, prolamine, trolamine, carbonate, phosphate (e.g., monopotassium phosphate, disodium phosphate), and mixtures of two or more thereof.

The pH of the compositions should be maintained from about 3.0 to about 10.0. Compositions having a pH of less than about 3.0 or greater than about 10.0 can increase the risk of irritating the nasal mucosa of the patient. Further, it is preferable that the pH of the compositions be maintained from about 3.0 to about 9.0. With respect to the non-aqueous nasal formulations, suitable forms of buffering agents can be selected such that when the formulation is delivered into the nasal cavity of a mammal, selected pH ranges are achieved therein upon contact with, e.g., a nasal mucosa.

The solubilizing agent for use in the compositions of the invention can be any known in the art, such as carboxylic acids and salts thereof. Exemplary carboxylic acid salts include acetate, gluconate, ascorbate, citrate, fumurate, lactate, tartrate, malate, maleate, succinate, or mixtures of two or more thereof.

The viscosity of the compositions of the present invention can be maintained at a desired level using a pharmaceutically acceptable thickening agent. For example, the viscosity may be at least 1000 cps; from about 1000 to about 10,000 cps; from about 2000 cps to about 6500 cps; or from about 2500 cps to about 5000 cps. Thickening agents that can be used in accordance with the present invention include, for example, methyl cellulose, xanthan gum, carboxymethyl cellulose, hydroxypropyl cellulose, carbomer, polyvinyl alcohol, alginates, acacia, chitosans, and mixtures of two or more thereof. The concentration of the thickening agent will depend upon the agent selected and the viscosity desired. Such agents can also be used in a powder formulation.

The nasally administrable compositions can also include a humectant to reduce or prevent drying of the mucus membrane and to prevent irritation thereof. Suitable humectants that can be used include, for example, sorbitol, mineral oil, vegetable oil and glycerol; soothing agents; membrane conditioners; sweeteners; and mixtures of two or more thereof. The concentration of the humectant will vary depending upon the agent selected. In one embodiment, the humectant can be present in the nasal delivery system in a concentration ranging from about 0.01% to about 20% by weight of the composition.

In other embodiments, the nasal delivery system can further comprise surfactants which enhance the absorption of the proton pump inhibitor. Suitable surfactants include non-ionic, anionic and cationic surfactants. Exemplary surfactants include oleic acid, polyoxyethylene derivatives of fatty acids, partial esters of sorbitol anhydride, such as for example, Tweens (e.g., Tween 80, Tween 40, Tween 20), Spans (e.g., Span 40, Span 80, Span 20), polyoxyl 40 stearate, polyoxy ethylene 50 stearate, fusieates, bile salts, octoxynol, and mixtures of two or more thereof. Exemplary anionic surfactants include salts of long chain hydrocarbons (e.g., C₆₋₃₀ or C₁₀₋₂₀) having one or more of the following functional groups: carboxylates; sulfonates; and sulfates. Salts of long chain hydrocarbons having sulfate functional groups are preferred, such as sodium cetostearyl sulfate, sodium dodecyl sulfate and sodium tetradecyl sulfate. One particularly preferred anionic surfactant is sodium lauryl sulfate (i.e., sodium dodecyl sulfate). The surfactants can be present in an amount from about 0.001% to about 50% by weight, or from about 0.001% to about 20% by weight.

The pharmaceutical compositions of the invention may further comprise an isotonicity agent, such as sodium chloride, dextrose, boric acid, sodium tartrate or other inorganic or organic solutes.

The nasal pharmaceutical compositions of the invention can optionally be used in combination with a pH adjusting agent. Exemplary pH adjusting agents include sulfuric acid, sodium hydroxide, hydrochloric acid, and the like.

To extend shelf life, preservatives can be added to the nasally administrable compositions. Suitable preservatives that can be used include benzyl alcohol, parabens, thimerosal, chlorobutanol, benzalkonium chloride, or mixtures of two or more thereof. Preferably benzalkonium chloride is used. Typically, the preservative will be present in a concentration of up to about 2% by weight. The exact concentration of the preservative, however, will vary depending upon the intended use and can be easily ascertained by one skilled in the art.

Other ingredients which extend shelf life can be added such as for example, antioxidants. Some examples of antioxidants include sodium metabisulfite, potassium metabisulfite, ascorbyl palmitate and the like. Typically, the antioxidant will be present in the compositions in a concentration of from about 0.001% up to about 5% by weight of the total composition.

Other optional ingredients can also be incorporated into the nasal delivery system provided that they do not interfere with the action of the proton pump inhibitor or significantly decrease the absorption of the proton pump inhibitor across the nasal mucosa.

The nasal delivery systems can be made following the processes described in, for example, U.S. Pat. Nos. 6,451,848, 6,436,950, and 5,874,450, and WO 00/00199, the disclosures of which are incorporated by reference herein in their entirety.

Each of the patents and publications cited herein are incorporated by reference herein in their entirety.

It will be apparent to one skilled in the art that various modifications can be made to the invention without departing from the spirit or scope of the appended claims.

What is claimed is:

1. A method for treating or preventing cystic fibrosis, a chronic ear infection, radiation therapy-induced emesis, emesis-induced tooth decay, bruxism, motion sickness, an NSAID-induced gastrointestinal disorder, a migraine, or exercise-induced gastroesophageal reflux disease in a patient in need thereof comprising administering a therapeutically effective amount of at least one proton pump inhibitor.

2. The method of claim 1, wherein the proton pump inhibitor is omeprazole or a pharmaceutically acceptable salt thereof, lansoprazole or a pharmaceutically acceptable salt thereof, esomeprazole or a pharmaceutically acceptable salt thereof, pantoprazole or a pharmaceutically acceptable salt thereof, leminoprazole or a pharmaceutically acceptable salt thereof, timoprazole or a pharmaceutically acceptable salt thereof, tenatoprazole or a pharmaceutically acceptable salt thereof, disulprazole or a pharmaceutically acceptable salt thereof, RO 18-5362 or a pharmaceutically acceptable salt thereof, IY 81149 or a pharmaceutically acceptable salt thereof, or 3-butyl-4-(2-methylphenylamino)-8-(2-hydroxyethoxy)-quinoline or a pharmaceutically acceptable salt thereof.

3. The method of claim 1, wherein the proton pump inhibitor is rabeprazole, a pharmaceutically acceptable salt thereof, a stereoisomer thereof, or a pharmaceutically acceptable salt of a stereoisomer thereof.

4. The method of claim 1, wherein the proton pump inhibitor is administered in an amount of 10 to 30 mg per day,

5. The method of claim 1, wherein the patient is a child.

6. The method of claim 1, wherein the method of treating cystic fibrosis further comprises administering albuterol, theophylline, ipratropium, guaifenesin, dnase, n-acetylcysteine, triamcinolone, flunisolide, fluticasone, beclomethasone, prednisone, methylprednisone, ibuprofen, montelukast, cromolyn, ciprofloxacin, co-trimoxazole, tobramycin, cephalexin, colistin, dicloxacillin, azithromycin, vitamins, pancrelipase, docusate, casanthranol/docusate, omeprazole, ranitidine, loratadine, cetirizine, fexofenadine, or a combination of two or more thereof.

7. The method of claim 1, wherein the method of treating the chronic ear infection further comprises administering amoxicillin; amoxicillin and clavulanate potassium; cefpodoxime proxetil; ceftriaxone; cefuroxime; or trimethoprim/sulfamethoxazole.

8. The method of claim 1, wherein the method for treating motion sickness further comprises administering scopolamine, promethazine hydrochloride, dimenhydrinate, diphenhydramine, cyclizine, buclizine, or meclizine.

9. The method of claim 1, wherein the method for treating or preventing an NSAID-induced gastroinstestinal disorder further comprising administering celecoxib, rofecoxib, valdecoxib, ibuprofen, acetaminophen, aspirin, naproxen, acetaminophen/aspirin/caffeine, ketorolac, ketoprofen, diflunisal, salsalate, salicylate, salicylamide, thiosalicylate, trisalicylate, mesalamine, sulfasalazine, methylsalicylate, phenylbutazone, oxyphenbutazone, antipyrine, aminopyrine, dipyrone, azapropazone, phenacetin, indomethacin, sulindac, mefenamic, meclofenamic, flufenamic, tolfenamic, etofenamic, tolmetin, naproxen, flurbiprofen, fenoprofen, fenbufen, pirprofen, oxaprozin, indoprofen, tiaprofenic acid, piroxicam, ampiroxicam, tenoxicam, tolmetin, meloxicam, tenidap, diclofenac, diclofenac/misoprostol, sulindac, etodolac, nabumentone, or a combination of two or more thereof.

10. A method for treating or preventing gastroesophageal reflux in a patient receiving anesthesia comprising administering a therapeutically effective amount of at least one proton pump inhibitor.

11. The method of claim 10, wherein the proton pump inhibitor is administered parenterally.

12. The method of claim 10, wherein the proton pump inhibitor is administered before surgery.

13. The method of claim 10, wherein the proton pump inhibitor is administered during surgery.

14. The method of claim 10, wherein the proton pump inhibitor is administered after surgery.

15. The method of claim 10, wherein the proton pump inhibitor is omeprazole or a pharmaceutically acceptable salt thereof, lansoprazole or a pharmaceutically acceptable salt thereof, esomeprazole or a pharmaceutically acceptable salt thereof, pantoprazole or a pharmaceutically acceptable salt thereof, leminoprazole or a pharmaceutically acceptable salt thereof, timoprazole or a pharmaceutically acceptable salt thereof, tenatoprazole or a pharmaceutically acceptable salt thereof, disulprazole or a pharmaceutically acceptable salt thereof, RO 18-5362 or a pharmaceutically acceptable salt thereof, IY 81149 or a pharmaceutically acceptable salt thereof, or 3-butyl-4-(2-methylphenylamino)-8-(2-hydroxyethoxy)-quinoline or a pharmaceutically acceptable salt thereof.

16. The method of claim 10, wherein the proton pump inhibitor is rabeprazole, a pharmaceutically acceptable salt thereof, a stereoisomer thereof, or a pharmaceutically acceptable salt of a stereoisomer thereof.

17. The method of claim 10, wherein the proton pump inhibitor is administered in an amount of 10 to 30 mg per day.

18. A method for treating or preventing gastroesophageal reflux disease in a human infant in need thereof comprising administering to the human infant a therapeutically effective amount of at least one proton pump inhibitor.

19. The method of claim 18, wherein the proton pump inhibitor is omeprazole or a pharmaceutically acceptable salt thereof, lansoprazole or a pharmaceutically acceptable salt thereof, esomeprazole or a pharmaceutically acceptable salt thereof, pantoprazole or a pharmaceutically acceptable salt thereof, leminoprazole or a pharmaceutically acceptable salt thereof, timoprazole or a pharmaceutically acceptable salt thereof, tenatoprazole or a pharmaceutically acceptable salt thereof, disulprazole or a pharmaceutically acceptable salt thereof, RO 18-5362 or a pharmaceutically acceptable salt thereof, IY 81149 or a pharmaceutically acceptable salt thereof, or 3-butyl-4-(2-methylphenylamino)-8-(2-hydroxyethoxy)-quinoline or a pharmaceutically acceptable salt thereof.

20. The method of claim 18, wherein the proton pump inhibitor is rabeprazole, a pharmaceutically acceptable salt thereof, a stereoisomer thereof, or a pharmaceutically acceptable salt of a stereoisomer thereof.
